(19) United States
(12) Patent Application Publication
(10) Pub. No.: US 2024/0008840 A1
(43) Pub. Date: Jan. 11, 2024

(54) ULTRASOUND TRANSDUCER PROBE WITH MULTI-ROW ARRAY ACOUSTIC STACKS AND ULTRASOUND IMAGING SYSTEM

(71) Applicant: IMSONIC MEDICAL CHINA, INC., Kunshan (CN)

(72) Inventor: Xiaohui HAO, Kunshan (CN)

(21) Appl. No.: 18/004,138

(22) PCT Filed: May 9, 2020
(86) PCT No.: PCT/CN2020/089400

§ 371 Date: Jan. 3, 2023

(51) Int. Cl. A61B 8/08; A61B 8/00; B06B 1/06

(52) U.S. Cl. CPC A61B 8/0841; A61B 8/4488; B06B 1/067; B06B 2201/76

(57) ABSTRACT

An ultrasound transducer probe with multi-row array acoustic stacks comprises an central acoustic stack and two side acoustic stacks, the central acoustic stack has an inverted trapezoidal shape backing, and the two side acoustic stacks are mounted on each of the two elevation direction sides of the central acoustic stack with an outward tilted angle α, this angle α ranges from 0 to 30 degrees. When all the acoustic stacks are electronically powered in the same time, an acoustic field with enlarged elevation section will be created to facilitate needle imaging.

TECHNICAL FIELD

The present invention relates to a real time ultrasound imaging system, and more particularly it relates to an ultrasound transducer probe with multi-row array acoustic stacks and an ultrasound imaging system.

### BACKGROUND ART

In tissue biopsy and interventional surgery, high frequency linear probes or curve linear low frequency probes are normally used for biopsy needle and interventional needle/wire inserting ultrasound guidance. In China and U.S., many clinicians don't use biopsy guide mounted on probe for needle guidance any more, rather, they depend on their experience—the subtle feeling of their fingers to the force passed from the needle tip when it is moving inside tissues, and the live image on the screen. They typically hold the transducer on top of the skin surface above the biopsy or interventional surgery tissue with one hand, use the other hand to hold and manipulate the needle under real time ultrasound monitoring. This operation is so difficult that generally only the most experienced ultrasound physicians can do it. The major difficulty encountered here is that, often, physicians can't easily find the needle body or needle tip during the operation. This is because that the state-of-art ultrasound transducers used for the procedure guidance, either a high frequency linear transducer working at a high center frequency, e.g., 6-12 Mhz, or a lower frequency curve linear transducer working around 2-5 MHz, etc. all have a thin ultrasound acoustic imaging field that is not so friendly for needle capturing.

The effective acoustic field, defined as the field with typically 20 dB lower intensity from the maximum is in a thin wall shape 3D field, with the elevation direction as the thinner direction. The azimuth direction is along the array element direction in which the sound beams moves and the elevation is defined as the direction perpendicular to azimuth direction. As during the biopsy or interventional surgery procedure, the needle often is in parallel or outside to the thin wall shape ultrasound beam, thus, hard to be captured by the acoustic volume, not visible in the formed ultrasound image. This then puts a very high skill requirement on operating physician. For inexperienced physician, this could be a big trouble especially since the biopsy/interventional surgery procedure itself is quite intense and often causes nervousness.

An example of such a technique is given in WO 2018/054969, which discloses an ultrasound imaging system, and which comprises an ultrasound transducer array comprising a plurality of ultrasound transducer tiles, each of the said tiles having an independently adjustable orientation such as to conform an ultrasound transmitting surface to a region of a body including a foreign object such as a pacemaker, a stent, or an interventional tool. Using a known spatial arrangement of a plurality of features of the foreign object, the respective ultrasound images generated by the ultrasound transducer tiles are registered in order to generate a composite image, in which the position and orientation of the foreign object in the individual images is superimposed. The position and orientation of an interventional tool may be determined for each image using object recognition algorithms or using acoustic feedback information provided by at least three ultrasound sensors arranged in a known spatial arrangement on the interventional tool. However, such an ultrasound imaging system relies on its independent adjustable orientation transducer tiles, which is a different solution from a transducer with arrays fixedly installed. In addition, it requires the ultrasound imaging system to cooperate with a biopsy needle mounted ultrasound sensor to work efficiently.

### SUMMARY OF THE INVENTION

The present invention aims to overcome the deficiency of that physicians can't easily find the needle body or needle tip during the operation of a biopsy or interventional needle often cannot be found in an ultrasonic image in the prior art.

In a first aspect of the present invention, an ultrasound transducer probe with multi-row array acoustic stacks, comprising:
a central acoustic stack for central row element array, fastened on support structure;
side acoustic stacks for side row element arrays, mounted on each of the two elevation direction sides of the central acoustic stack with an outward tilted angle α, wherein the angle α ranges from 0 to 30 degrees;
the central acoustic stack and the side acoustic stacks are used for transmitting and receiving ultrasonic signals; and
a shell, disposed to house all of the acoustic stacks and support structure.

In a further aspect of the present invention, an ultrasound imaging system, comprising:
an user interface, used for information interaction with the processing system of the ultrasound imaging system;
an ultrasound transducer probe that is electrically connected to the processing system, the ultrasound transducer probe comprises:
an central acoustic stack for central row element array, fastened on support structure; and
side acoustic stacks for side row element arrays, mounted on each of the two elevation direction sides of the central acoustic stack with an outward tilted angle α, this angle α ranges from 0 to 30 degrees; the central acoustic stack and the side acoustic stacks are used for transmitting and receiving ultrasonic signals; and
a shell, disposed to house all of the acoustic stacks and support structure.

Preferred embodiment of the invention are defined in the dependent claims. It shall be understood that the claimed ultrasound transducer probe of the ultrasound imaging system has similar and/or identical preferred embodiments as the claimed ultrasound transducer probe and as defined in the dependent claims.

In a preferred embodiment, the central acoustic stack has an inverted trapezoidal shape backing with a tilted angle β, and the angle a matches the angle β.

In a preferred embodiment, all the acoustic stacks are put together with gap in between such that the flex circuitry boards of all acoustic stacks can go through.

In a preferred embodiment, the support structure includes a support shelf and frames, the three acoustic stacks are held together by the frames, and the frames are provided at both ends of the acoustic stacks and fastened on the top of the support shelf by screw sets.

In a preferred embodiment, the support shelf and acoustic stacks are set with gap in between for the flex circuitry boards to go through.

In a preferred embodiment, the shell includes body shell and head shell, the said support shelf is mounted on body shell, and the head shell is used to housing all the acoustic stacks.

In a preferred embodiment, the ultrasound transducer probe further comprises a lens at least disposed on top of the central acoustic stack.

In a preferred embodiment, the lens is disposed on top of all the acoustic stacks and fastened on the head shell.

In a preferred embodiment, the central acoustic stack includes a first matching layer, a second matching layer and a piezoelectric layer sequentially, the underneath of piezoelectric layer is metalized to form ground electrode and signal electrode that connected to the flex circuitry board.

In a preferred embodiment, an acoustic backing layer is disposed below the piezoelectric layer and flex circuitry board, and the flex circuitry board extends downward along the two sides of the acoustic backing layer.

In a preferred embodiment, each of the side acoustic stacks includes a first matching layer, a second matching layer and a piezoelectric layer sequentially, the underneath of piezoelectric layer is metalized to form ground electrode and signal electrode connected to the flex circuitry board.

In a preferred embodiment, an acoustic backing layer is disposed below the piezoelectric layer and flex circuitry board, and the flex circuitry board extends downward along the two sides of the acoustic backing layer. The side acoustic stacks have an acoustic structure similar to the central acoustic stack.

In a preferred embodiment, the lower end of the acoustic backing layer of the central acoustic stack forms the said inverted trapezoidal shape, and the acoustic backing layers of the side acoustic stacks are arranged to match the acoustic backing layer of the central acoustic stack.

In a preferred embodiment, each of the side acoustic stacks has the same number of array elements as the central acoustic stack.

In a preferred embodiment, each of the side acoustic stacks has the same height of array element as the central acoustic stack.

In a preferred embodiment, each side acoustic stack is provided with an independent control circuit , one or two control buttons for the control circuit are located on the body shell.

In a preferred embodiment, two or more side acoustic stacks are mounted on each of the two elevation sides of the central acoustic stack.

The present invention introduces a special ultrasound transducer probe structure design, in which two extra side acoustic stacks for side row element array are added to the sides of the conventional one central acoustic stack for central row element array. These added side acoustic stacks are either slightly tilted outward or not tilted in order to form an enlarged effective acoustic field which is thicker in elevation direction for each transmit and receive ultrasound beams. As a result, when all acoustic stacks are turned on, the formed acoustic field, with a near hyperboloid cross section in elevation, gains more thickness in elevation, such that the needle can be captured in a much easier way—because things that fall inside the effective range of the acoustic field can be captured. When the side acoustic stacks are outward tilted, the central acoustic stack is having an inverted trapezoidal shape backing to allow the room for the side acoustic stacks.

The structural arrangement of the present invention comprehensively considers the sound field effect, spatial arrangement, circuit layout and other factors of the acoustic stacks, while achieving a better visual effect, the overall spatial layout in the probe is more reasonable. This new designed structure reduces the probe volume and is more convenient for doctors to use.

### BRIEF DESCRIPTION OF THE DRAWINGS

FIG. 1 is an illustration of a linear array probe having multi-row array acoustic stacks.

FIG. 2 shows the elevation cross section of the ultrasound transducer probe.

FIG. 3 shows the 3D view of the structure of multi-row array acoustic stacks.

FIG. 4 shows the azimuth cross section of the structure of central acoustic stack and its position and connection structure.

FIG. 5 shows the structure of the central acoustic stack.

FIG. 6 shows the structure of a side acoustic stack.

FIG. 7 gives the explosion assembly drawing of the transducer acoustic stack structure.

FIG. 8a shows the support shelf formed by two separated support feet.

FIG. 8b shows two support feet connected by a crossbar below the top end of the support feet.

FIG. 9 illustrates a lens arrangement for the central acoustic stack.

FIG. 10 illustrates the outlook of the multi-row array ultrasound transducer probe shell with a button.

FIG. 11 shows the electronic controls of the three array acoustic stacks

FIG. 12 shows the effective acoustic field generated by conventional linear array transducer.

FIG. 13 shows the effective acoustic fields generated by the innovated multi-row arrays transducer, and the biopsy needle in the field of side row array transducer.

FIG. 14 shows the simulated acoustic field contour for conventional linear array transducer and invented multi-row arrays transducer.

### DETAILED DESCRIPTION OF THE EMBODIMENTS

For further understanding of the present invention, the present invention is described in detail with reference to the drawings and embodiments.

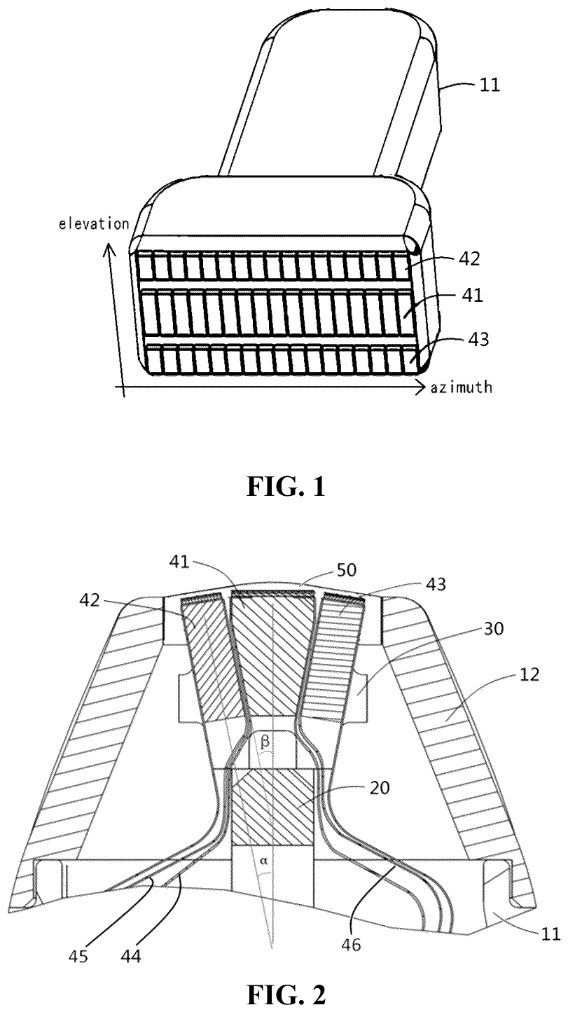

FIG. 1 illustrates an example of a multi-row linear array probe that enhances the visualization of a puncture needle. The probe comprising: an central acoustic stack 41 for central row element array; a side acoustic stack 42 for side row element array, mounted on a left side of the central acoustic stack 41, and a side acoustic stack 43 mounted on a right side of the central acoustic stack 41. All the acoustic stacks are used for transmitting and receiving ultrasonic signals.

In a coordinate system of FIG. 1, an azimuth direction is the element arrangement direction in an array, and an elevation direction is perpendicular to the element arrangement direction, and also refers to a direction perpendicular to the side wall of the probe. A plurality of the element arrays is distributed along the elevation direction. The invented ultrasound transducer is done as a high frequency linear ultrasound transducer, in which, except the traditional central row element array as in a conventional ultrasound transducer, two extra rows of element arrays in elevation direction are added into the ultrasound transducer.

An example embodiment of such an ultrasound transducer probe is described with more details with the aid of FIG. 2, which schematically shows an illustration of the elevation cross section of the invented ultrasound transducer probe structure. The embodiment achieves a better needle visualization by increasing the thickness of the ultrasound imaging field through a new transducer structure design, in which, two side acoustic stacks 42-43 are mounted on each of the two elevation direction sides of the central acoustic stack 41 with an outward tilted angle α, and the angle α ranges from 0 to 30 degrees, such as 10/15/18/22 degrees.

As shown in FIG. 2, the central acoustic stack 41 has an inverted trapezoidal shape backing with a tilted angle Δ, and the angle a matches the angle Δ, the angle Δ can be bigger or a bit smaller than angle α. They can also be equal to each other. This is to allow the side acoustic stacks 42-43 for the two side row element arrays to be mounted on the side of the central acoustic stack 41 with an outward tilted angle α. The three acoustic stacks are fastened on a support structure including a support shelf 20 and frames 30, and the three acoustic stacks are held together by frames 30 provided at both ends of the acoustic stacks, and the frames 30 are fastened on the top of the support shelf 20 by screws. A shell including a body shell 11 and a head shell 12 is added to house all the acoustic stacks and the support structure, the support shelf 20 is mounted on the body shell 11, a head shell 12 is used to house all the acoustic stacks.

Furthermore, some lens material is filled on top of the three acoustic stacks to form lens 50, and the lens mounted on the head shell.

In an embodiment, all the acoustic stacks are put together with gap in between such that flex circuitry boards 44, 45, and 46 of each acoustic stack 43, 42 and 41 can go through. The flex circuitry boards 44, 45 and 46 are used to transmit or receive signals or transmit electric power for some components in acoustic stacks.

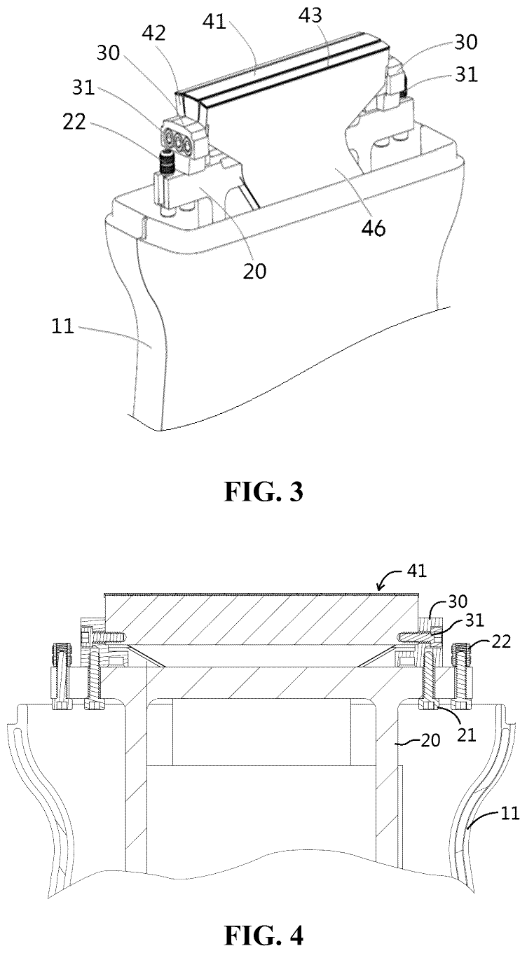

FIG. 3 further gives a 3D view of the ultrasound transducer probe to illustrate the support structure. In FIG. 3, the central acoustic stack 41 and two side acoustic stacks 42-43 are held together by frames 30. The support shelf 20 and frames can be made by metal material, and the support shelf 20 is fixed to the body shell 11 by screw sets 21, two frames 30 are fastened on the top of the support shelf 20 by screw sets 21.

In a preferred embodiment, the acoustic stacks are fastened between the two frames 30 by screw sets 31. And the screw sets 31 are screwed into the acoustic stacks in azimuth direction so that it won't shake, and it is easy to remove and install the acoustic stacks.

FIG. 4 further gives a section which is perpendicular to azimuth direction and through the center of the central acoustic stack 41 to better show the holding structure. In the figure, it can be seen that central acoustic stack 41 is held by metal frames 30 through fasten screw sets 31 from two sides.

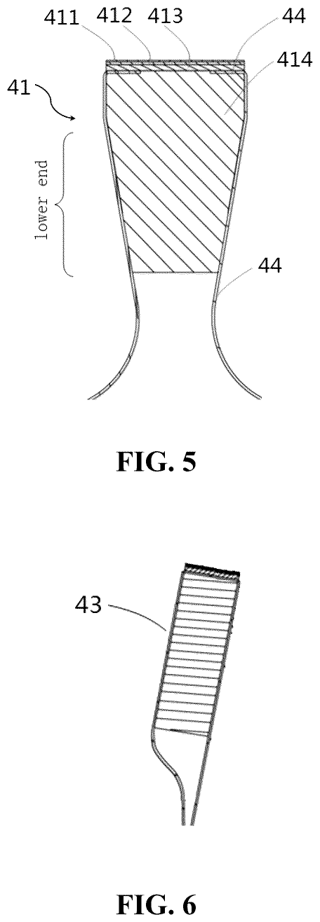

FIG. 5 illustrates the details of the central acoustic stack 41. It has a first matching layer 411, a second matching layer 412, and then underneath the second matching layer 412, a piezoelectric layer 413. The first and second matching layers typically are made by epoxy and are used to maximize the acoustic wave signal strength from the piezoelectric layer to human tissue. The piezoelectric layer 413 has a much larger acoustic impedance than the human tissue, which is problematic in transmitting energy directly into tissue due to large amount reflection. To avoid this low efficiency, the first and second matching layer 411 and 412 have acoustic impedance value in between human tissue and piezoelectric layer, e.g., matching layer 412 may have an acoustic impedance 7 MRayls and matching layer 411 may have an acoustic impedance 3 MRayls, these two matching layers gradually drop the acoustic impedance difference in between piezoelectric layer and human tissue, thus reducing the amount of reflected energy at the surfaces of different layers, allowing much high energy transmission efficiency.

In a preferred embodiment, both the first and second matching layers have a thickness of ¼ wavelength of the probe center frequency. The piezoelectric layer has a thickness of ½ wavelength of the center frequency, and can be made from piezo-ceramic, piezoelectric single crystal, piezoelectric composite material. The underneath of the piezoelectric layer 413 is metalized to form ground electrodes and signal electrodes such that flex circuitry board 44 which is bonded under the piezoelectric layer 413 can connect with the ground electrodes and signal electrodes of each acoustic element. Below the piezoelectric layer 413 and flex circuitry board 44 is an acoustic backing layer 414. The acoustic backing layer 414 has an inverted trapezoidal shape, with its bigger size end bonding with piezoelectric layer 413. The acoustic backing layer 414 can be constructed with an epoxy and alumina powder, or with epoxy and Tungsten powder, or with epoxy, alu mina and Tungsten powder and some other materials. The backing layer 414 is used to give mechanical support to the acoustic piezoelectric layer 413 and other layers above 413, to provide maximal efficiency in the electromechanical coupling, and to prevent reverberation. Typically, the backing layer 414 can have an acoustic impedance range from 5 MRayls to 20MRalys or higher.

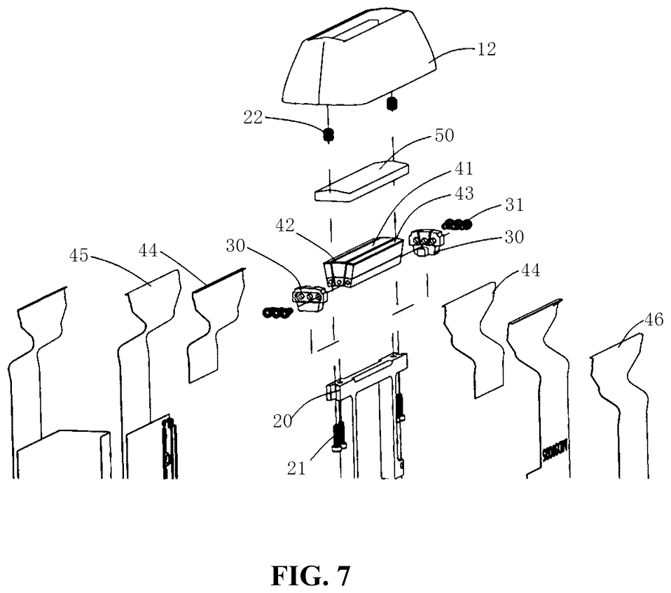

The flex circuitry board 44 has its signal traces and ground traces split into two groups, typically the even group and the odder group. The two groups of signal traces and ground traces go down as the flex circuitry board 44 extends downward along the two sides of the backing layer 414. The side acoustic stacks 42-43 have similar acoustic structure with the central acoustic stack 41, but the acoustic backing layer in the side acoustic stacks 42-43 has a different shape as shown in FIG. 6.

The acoustic elements in the side acoustic stack 42/43 may be made in the same material as the elements in the central acoustic stack 41, e.g., made by piezo-ceramic or piezo-single crystal (PMN-PT, PIN-PT) material, thick film of piezo-ceramic or piezo-single crystal (PMN-PT, PIN-PT) material, or they can be made by 1-3 composites of piezo-ceramic or piezo-single crystal (PMN-PT, PIN-PT) material. The 1-3 composites material may include regular post structure, such as square post, triangular post or random structure. Furthermore, the multi-row array ultrasound transducer in whole can be made using cMUT technology which is basically silicon chips.

FIG. 7 gives the explosion assembly drawing of the transducer acoustic stack structure. It can be seen that the acoustic stack 41, 42 and 43 are fastened between the two frames 30 by screw sets 31, while frames 30 are fixed to structure 20 by screw sets 21 and nuts 22. The flex circuitry boards 44 which are for sending and receiving signals of acoustic stack 41, the flex circuitry boards 45 which are for sending and receiving signals of acoustic stack 42, and the flex circuitry boards 46 which are for sending and receiving signals of acoustic stack 43, all pass through the gap between support shelf 20 and the acoustic stack 41, 42, 43 to the electronic control boards below. Lens 50 is glued to the surface of acoustic stack 41, 42 and 43. The head shell 12 is then put on top of the lens 50 to house the whole acoustic stack head structure.

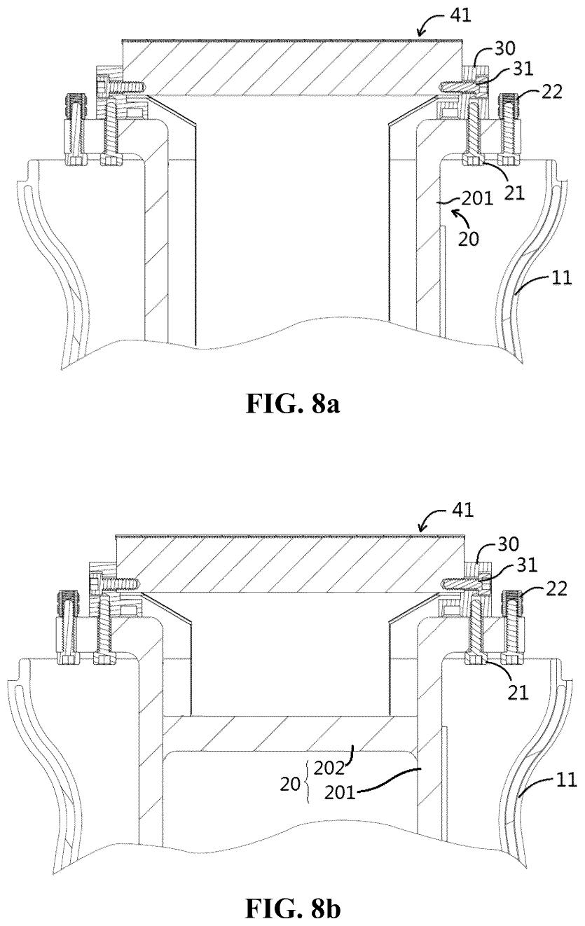

FIG. 8a illustrates an example in which the support shelf 20 is formed by two separated support feet 201 fixed to the body shell 11. And FIG. 8b gives an example in which two support feet 201 are connected by a crossbar 202 ,which is below the top end of the support feet. In contrast with the two examples in FIG. 8a and 8b, the structure of the support frame 20 in FIG. 4 is a preferred embodiment. FIG. 4 shows that a crossbar is set on top of the support feet to obtain a stronger structure.

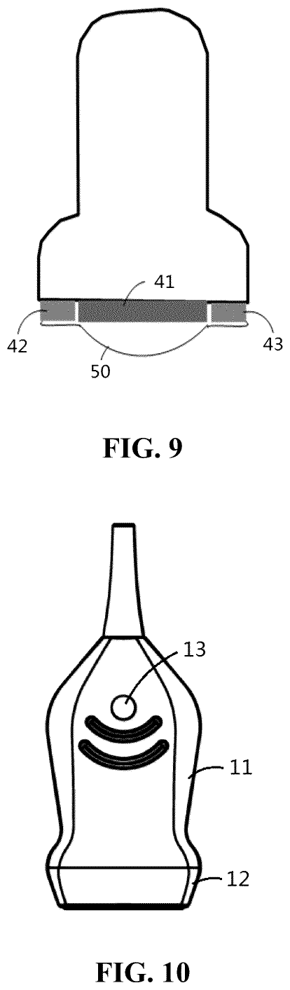

FIG. 9 illustrates an example in which a lens arrangement is just for the central acoustic stack. Further detail improvements may include a special lens which only covers the main row elements in central row element array and leave the elements of two side row element arrays uncovered, so the effect acoustic field they created varies more and results in more thickness in elevation direction.

FIG. 10 gives an outlook of the probe body, where the control button 13 is shown.

Due to the fundamental that the image pixel at certain depth and lateral position is formed by the summation of the tissue signals of the resolution cell volume centered at that spatial location, a thick elevation volume often results in lower image spatial resolution and a more haze like image, thus it worsens contrast resolution as more tissue is integrated inside this volume and contributes to the final reflected signal.

To avoid the degradation of image resolution, often the contrast resolution, in normal imaging with this specially designed ultrasound transducer, a separate control is added such that only when needed, the arrays in two side rows will be turned on to form a thick acoustic field in elevation direction. Each of the three element arrays can be powered and electronically or manually controlled separately.

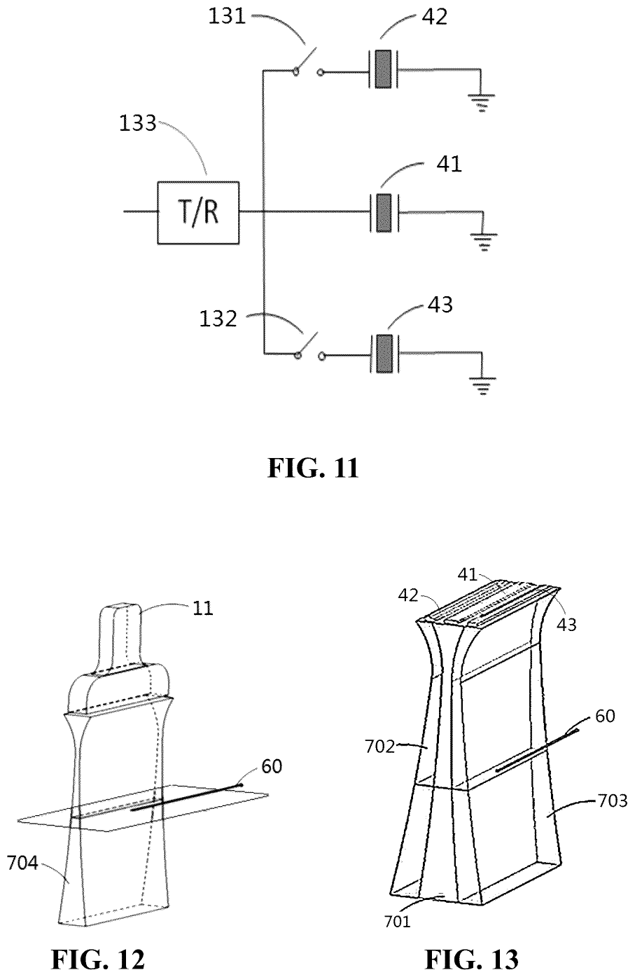

FIG. 11 shows the diagram of electronic control of the three rows of element arrays. In the figure, waveform send from the system through T/R switch 133 will be delivered to the central element array directly. While in the mean time, switch controls 131-132 guard the waveform transfer to and echo receiving from side row element arrays. Switch controls 131-132 could be electronic switches controlled by the system, or could be buttons that operator can push to turn on or off. When both switch controls are turned on, the side acoustic stacks 42-43 will be connected to T/R switch 133, thus, setup the same signal paths as the signal path for the central acoustic stack 41. Transmit signals will go out from T/R switch 133 to array acoustic stacks 41-43 simultaneously. The echo received from tissue by the acoustic stacks 41-43 will be naturally summed at T/R switch 133 then sent to the processing system. During the operating procedure, clinicians could turn on the two side acoustic stacks 42-43 to form a thicker acoustic field in elevation direction for better needle visualization and turn them off when needle is found and clearer image is preferred.

FIGS. 12 and 13 give a demonstration of the benefit of this new ultrasound transducer probe structure. In FIG. 12, a regular high frequency linear probe is used to monitor biopsy needle 60 but missed the needle. In this case, this transducer transmits multi-ultrasound beams at a high center frequency, e.g., 10-12 MHz, from left to right, forms a curved wall shape like effective acoustic volume 704, with a hyperboloid section in the plane that is perpendicular to the azimuth direction. This acoustic volume, identified as acoustic field of this probe, defined by the signal strength at −30 dB from the maximum acoustic intensity, is the volume resulted from the imaging beams during real time scanning.

Objects in the acoustic volume 704, such as tissue, bones, needles, wires, etc. can be clearly defined in ultrasound image. The acoustic volume 704 has a pretty thin slice thickness in elevation direction. If the needle 60, full or part of it, falls in this acoustic volume 704, it will show up in the real time image. As during the biopsy or interventional surgery procedure, the needle often is in parallel and outside to this thin wall like acoustic volume 704, e.g., it is on the plane but falling outside the acoustic volume 704, as a result it can't be captured by the acoustic volume 704, thus not visible in the formed ultrasound image. This could be a serious issue to an inexperienced clinician.

As a comparison, in FIG. 13, the three element arrays, when combined together, can form an acoustic field with much larger elevation width compared to only the central element array. This widened acoustic field, where the two extra rows of element arrays, when turned on, will create extra acoustic fields 701-703, shown as the shadowed regions in FIG. 12, beyond the acoustic field 701 generated by the central row of element array. These extra acoustic fields 702-703, when combined with the acoustic field 701, will form a much thicker acoustic field in elevation direction than acoustic field 701 alone. The formed thicker acoustic field has a larger hyperboloid section.

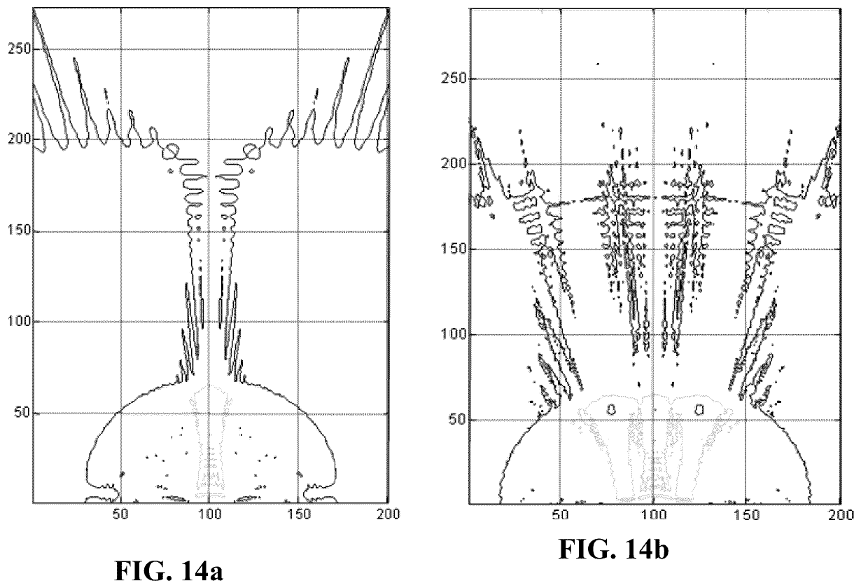

FIG. 14a shows the simulated acoustic fields for central row element array only, and FIG. 14b shows the simulated acoustic field when all three rows of element arrays turned on and formed the combined field where acoustic waves merged together. The −60 dB contour in FIG. 14b has a much larger width at every depth compared to the −60 dB contour in FIG. 13a.

In an embodiment, the side row element arrays and the central row element array may have the same number of array elements, and may have the same element pitch. In order to further improve the visual effect of the ultrasonic probe on the puncture and interventional surgery needles, in another embodiment, the side row element arrays may have different element pitch and even different numbers of elements. Therefore, the effective thickness of the acoustic field generated by the probe is increased as much as possible to enable the acoustic field to capture the puncture needle body parallel to the main direction of the acoustic field more easily.

As an example of an ultrasound imaging system, it includes the above-mentioned ultrasound transducer probe and an user interface used for information interaction with the processing system. In this system, a user manipulates the processing system through the user interface, so that the system enters into a needle head guidance working mode for tissue biopsy or interventional surgery.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The mere fact that certain measures are recited in mutually different dependent claims does not indicate that a combination of these measures cannot be used to advantage.

1. An ultrasound transducer probe with multi-row array acoustic stacks, comprising:
a central acoustic stack (41) for central row element array, fastened on support structure;
side acoustic stacks (42-43) for side row element arrays, mounted on each of the two elevation direction sides of the central acoustic stack (41) with an outward tilted angle α, wherein the angle α ranges from 0 to 30 degrees;
the central acoustic stack (41) and the side acoustic stacks (42-43) are used for transmitting and receiving ultrasonic signals; and
a shell, disposed to house all of the acoustic stacks and the support structure.

2. The ultrasound transducer probe of claim 1, wherein the central acoustic stack (41) has an inverted trapezoidal shape backing with a tilted angle β, and the angle a matches the angle β.

3. The ultrasound transducer probe of claim 2, wherein all the acoustic stacks are put together with gap in between such that the flex circuitry boards (44-46) of all acoustic stacks can go through.

4. The ultrasound transducer probe of claim 3, wherein the support structure includes a support shelf (20) and frames (30), the three acoustic stacks are held together by the frames (30), and the frames (30) are provided at both ends of the three acoustic stacks and fastened on the top of the support shelf (20).

5. The ultrasound transducer probe of claim 4, wherein the support shelf (20) and acoustic stacks are set with gap in between for the flex circuitry boards (44-46) to go through.

6. The ultrasound transducer probe of claim 4, wherein the shell includes body shell (11) and head shell (12), the support shelf (20) is mounted on body shell (11), and the head shell (12) is used to house all the acoustic stacks.

7. The ultrasound transducer probe of claim 1, further comprising a lens (50) at least disposed on top of the central acoustic stack (41).

8. The ultrasound transducer probe of claim 7, wherein the lens (50) is disposed on top of all the acoustic stacks and fastened on the head shell (12).

9. The ultrasound transducer probe of claim 1, wherein the central acoustic stack (41) includes a first matching layer (411), a second matching layer (412) and a piezoelectric layer (413) sequentially, the underneath of piezoelectric layer (413) is metalized to form ground electrode and signal electrode that connected to the flex circuitry board (44).

10. The ultrasound transducer probe of claim 9, an acoustic backing layer (414) is disposed below the piezoelectric layer (413) and flex circuitry board (44), and the flex circuitry board (44) extends downward along the two sides of the acoustic backing layer (414).

11. The ultrasound transducer probe of claim 9, wherein each of the side acoustic stacks (42-43) includes a first matching layer (411), a second matching layer (412) and a piezoelectric layer (413) sequentially, the underneath of piezoelectric layer (413) is metalized to form ground electrode and signal electrode connected to the flex circuitry board (45-46).

12. The ultrasound transducer probe of claim 11, an acoustic backing layer is placed below the piezoelectric layer (413) and flex circuitry board (44), and the flex circuitry board (45-46) extends downward along the two sides of the acoustic backing layer.

13. The ultrasound transducer probe of claim 12, wherein the lower end of the acoustic backing layer (414) of the central acoustic stack (41) forms the inverted trapezoidal shape, and the acoustic backing layers of the side acoustic stacks (42-43) are arranged to match the acoustic backing layer (414) of the central acoustic stack (41).

14. The ultrasound transducer probe of claim 1, wherein each of the side acoustic stacks (42-43) has the same number of array elements as the central acoustic stack (41).

15. The ultrasound transducer probe of claim 1, wherein each of the side acoustic stacks (42-43) has the same height of array element as the central acoustic stack (41).

16. The ultrasound transducer probe of claim 1, wherein each side acoustic stack (42-43) is provided with an independent control circuit , one or two control buttons for the control circuit are located on the body shell (11).

17. The ultrasound transducer probe of claim 1, two or more side acoustic stacks are mounted on each of the two elevation sides of the central acoustic stack (41).

18. An ultrasound imaging system, comprising:
an user interface, used for information interaction with the processing system of the ultrasound imaging system;
an ultrasound transducer probe electrically connected to the processing system, the ultrasound transducer probe comprising:
a central acoustic stack (41) for central row element array, fastened on support structure; and
side acoustic stacks (42-43) for side row element arrays, mounted on each of the two elevation direction sides of the central acoustic stack (41) with an outward tilted angle α, the angle α ranges from 0 to 30 degrees; the central acoustic stack (41) and the side acoustic stacks (42-43) are used for transmitting and receiving ultrasonic signals; and
a shell, disposed to house all of the acoustic stacks and support structure.

19. The ultrasound transducer probe of claim 5, wherein the shell includes body shell (11) and head shell (12), the support shelf (20) is mounted on body shell (11), and the head shell (12) is used to house all the acoustic stacks.

20. The ultrasound transducer probe of claim 2, wherein the central acoustic stack (41) includes a first matching layer (411), a second matching layer (412) and a piezoelectric layer (413) sequentially, the underneath of piezoelectric layer (413) is metalized to form ground electrode and signal electrode that connected to the flex circuitry board (44).
